(19) United States
(12) Patent Application Publication
(10) Pub. No.: US 2007/0071727 A1
(43) Pub. Date: Mar. 29, 2007

(54) Melanocytes in therapy of neurodegenerative diseases

(76) Inventor: Mats J. Olsson, Uppsala (SE)

Correspondence Address: RISSMAN JOBSE HENDRICKS & OLIVERIO, LLP, ONE STATE STREET, SUITE 800, BOSTON, MA, 02109, US

(21) Appl. No.: 10/579,566

(22) PCT Filed: Nov. 15, 2004
(86) PCT No.: PCT/SE04/01663

§ 371 Date: May 12, 2006

Related U.S. Application Data
(60) Provisional application No. 60/520,122, filed on Nov. 14, 2003.
(60) Provisional application No. 60/520,123, filed on Nov. 14, 2003.

(51) Int. Cl. A61K 35/36; C12N 5/08

(52) U.S. Cl. 424/93.7; 435/371

(57) ABSTRACT

In vitro cultivation method for producing human melanocytes which are capable of melanin and/or L-dopa synthesis, and to various uses of said melanocyte(s). In one preferred aspect, said melanocyte(s) are used in an autologous implantation method in a patient suffering from a neurodegenerative disease, e.g. Parkinson's disease. In another aspect, a screening method is provided utilizing melanocyte(s) from said cultures, wherein substances are screened for which may in any way effect said melanocyte(s). Said substances may e.g., be neurotoxic, neuroprotective, or particularly lethal to melanocytes obtained from a patient suffering from Parkinson's disease. The invention also relates to other uses of said melanocytes, all of which are disclosed herein.

### FIELD OF THE INVENTION

The present invention relates to human melanocytes obtained by an in vitro cultivation method according to the present invention, as well as various uses of said human melanocytes.

In a preferred aspect of the invention, said melanocytes may be used in an implantation method in patients suffering from a neurodegenerative disease, e.g. Parkinson's disease, as well as in a screening method for identifying compounds suitable as drugs for the treatment of a neurodegenerative disease. Said melanocytes are also implicated in medical and further medical uses.

### BACKGROUND OF THE INVENTION

The Melanocyte

The melanocyte (pigment cell) is a nerve cell related cell, which during embryogenesis migrates from the dorsal part of the neural crest and populates in addition to the epidermis, also the matrix in the hair follicles, stria vascularis of the inner ear, iris and choriodea in the eye, the ganglia in the enteric plexa of the gut, lepto menigns, striatum and the substantia nigra of the brain.

In the human skin, nerve fibres are in direct touch with melanocytes through synapse-resembling points of contact (Hara M et al. Innervation of melanocytes in human skin. J Exp Med. 1996 Oct. 1; 184(4):1385-95), and it is also shown that melanocytes in the iris in primates and humans receive both adrenergic and cholinergic innervation.

Certain amphibians and reptiles have the ability to redistribute the pigment in the melanophores following a nerve-mediating signal from the optic nerve and in this way make the animal change the colour compatible with the surroundings. This is based on a well-established knowledge that begun several decades ago with Aaron B. Lerner et al. showing that melanocyte-stimulating hormone (MSH) activates and melatonin deactivates this pathway of signal transduction. The melanophores from the Amphibias and their ability to rapidly redistribute the pigment within the cells even under cell-culture conditions, has come into practical use in the screening of substances, which are activating or inactivating G-protein coupled 7TMS receptors (Lerner M R. Tools for investigating functional interactions between ligands and G-protein-coupled receptors. Trends Neurosci. 1994 April; 17(4):142-6).

There are a number of important steps and mechanisms involved in the formation and distribution of melanin (the pigment) in human skin. Tyrosinase is the most important and rate-limiting enzyme in the production of melanin. The tyrosinase is mainly coupled to the inside of the membrane in the melanosomes of the melanocytes (lysosome-related melanin-producing vesicles). A small domain of this enzyme sticks out through the melanosome and is receptive to activating or inhibiting stimuli from factors in cytoplasm of the melanocyte. When factors get coupled to this “external” domain, the confirmation of the internal unit is altered, with an activity change as a consequence.

The mature melanin-containing melanosomes do by way of Kinesin/ATP mediated movement migrate into the dendrites of the melanocytes and are thereafter taken up by keratinocytes, where the melanin is spread and hence the skin is getting darker.

In the melanogenesis (syntheses of melanin) the copper-dependent tyrosinase catalyses the formation of dopa from tyrosine, dopaquinone from dopa as well as the DHI-oligomeres from DHI (DiHydroxyIndoles) according to the pathway:

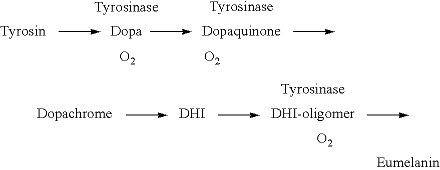

The ring formation of dopaquinone and hence the formation of dopachrome and DHI occurs with the help of divalent ions, which act as catalysers. Cu²⁺, Ni²⁺, Co²⁺ are needed for the formation of 5,6-DHI, and Zn²⁺ for the formation of 5,6-DHI-2-carboxyl acid. Both variations of DHI have the ability to form eumelanin, 5,6-DHI with the help of tyrosine and 5,6-DHI-2-carboxyl acid with the help of TRP-1 (see below).

There are two types of melanin, the black-brown eumelanin and the red-yellow phaeomelanin. Dopachrome can to a certain extent also be turned into eumelanin through a pair of sidesteps catalysed by TRP-2 (tyrosine-related protein-2), as well as TRP-1 (tyrosine-related protein-1). These enzymes are closely related to tyrosinase. Quantitatively, the major part of the eumelanin is formed by the tyrosine-regulated step and the general dogma has been that the melanin formed must go through the bottleneck of tyrosine→dopa→dopaquinone, which earlier was thought to be exclusively regulated by tyrosinase.

In contrast to the ring formation of dopaquinone, the formation of phaeomelanin occurs through following the addition of the sulphate-containing aminoacid cysteine, which gives rise to cysteinyidopa, which has the ability to turn into phaeomelanin. It is most likely the high sulphurcontent of phaeomelanin that is responsible for the yellowish-red, tone of the pigment.

The upregulation of the tyrosinase activity in the melanocytes of the skin is normally mediated by prostaglandins, leukotrienes, tromboxanes, interleukines, MSH (melanocyte stimulated hormone), steroids, FGF (fibroblast growth factor) and ultraviolet light.

Recently it has been shown that human epidermal melanocytes also express rather vast amount of tyrosine hydroxylase (TH), an enzyme important in the formation of neuromelanin in the CNS. In human epidermal melanocyte, TH is as tyrosinase coupled to the inner membrane of the melanosome (Marles L. K. et al. Regulation of Tyrosinase and tyrosine hydroxylase activities by α- and β-MSH. Pigment Cell Res. Vol 15, supplement 9, 2002, PO-010, page 59). TH does, as well as tyrosinase, oxidise tyrosine into L-dopa which after several other steps gives rise to melanin.

The melanocytes can only take up tyrosine through passive diffusion over the cell membrane, which is a slow transport mechanism and which is not giving the cell enough building material to support the synthesis of melanin. To get around this problem, the melanocyte express high levels of phenylalanine-hydroxylase (PH). With the help of PH, phenylalanine is rapidly converted into tyrosine in the cytoplasm of the cell. The outer membrane of the melanocyte contains vast amounts of active (ATP-driven) pumps for the rapid shunting of phenylalanine.

In children with phenylketoneurea (PKU) there is a mutation of the PH-gene, which in turn leads to reduced synthesis of epidermal and neural pigment which quite often has a neurological handicap as a consequence.

The importance of the melanocyte in hearing is obvious from the fact that when the areas around the ears are depigmented in people with Waardenburg syndrome and Piebaldism, because of a failure in melanoblast migration during the embryogenesis, the result is deafness.

The eumelanin has a protecting effect in the skin and contributes to antioxidation, buffering and eliminating of toxic chemicals. Eumelanin has strong antioxidative abilities and takes care of free radicals which are formed when the skin is subject to ultraviolet radiation and oxidative stress. Eumelanin is a polymer with negative charges that can attract and bind heavy metal-ions and toxic amines. In the skin, this melanin is further transferred to the keratinocytes, which after a while are then shed off as the stratum corneum in the continues renewal of the epidermis, and hereby act as a detoxifier of the skin.

L-dopa and Parkinson's Disease

The use of L-Dopa has radically changed the prognosis and strongly improved the quality of life for the majority of Parkinson's patients worldwide. However, there are still significant problems in the long-term prognosis for these patients. After 5-10 of years on L-dopa treatment, the medication is not as controllable of the disease as initially. This in turn often leads to total loss of muscle control and loss of speech.

It is apparent that the degeneration of brain tissue in the long run progresses beyond pigment-producing cells, which are the ones that are first to be lost. It is possible, that the secondary brain damage may be dependent on, or caused by, the loss of melanin-containing cells, which normally act detoxifying, antioxidative and buffering for their immediate environment. Melanins are polyanions, and substances with cationic properties, such as amines and metal ions are easily bound to melanin by ionic interaction (Mårs, U.: Melanogenesis as the Basis for Melanoma Target. In: Dept of Pharmaceutical Biosciences, Division of Toxicology. Uppsala University, Uppsala, 1998. ISBN 91-554-4246-3).

In the skin, the pigment-producing cells also have such protective functions and the eumelanin is proved to function as a buffer, entrapping metal ions and quench or scavenge photochemically generated free radicals (Jimbow, K.: Current update and trends in melanin pigmentation and melanin biology. Keio J Med 1995, 44, 9-18).

Dopamine is known to be cytotoxic and patients with Parkinson's disease, which are on medication for long periods with L-dopa, converted to dopamine in the tissue have, because of their loss of neuronal pigment cells also lost their ability to store and detoxify the substance in a diligent manner. Tyrosinase has shown to have a protective effect on dopamine cytotoxicity through the enzyme's ability to convert dopamine to dopaminequinone through oxidation, which in turn can be converted further to neuromelanin (J. Matsunaga et al.).

Data also reveal a TH-independent major pathway of peripheral dopamine synthesis in the skin melanocytes of young mice. The transient nature of this source of dopamine reflects a developmental switch in tyrosinase-dependent production of dopamine to production of melanin. (Eisenhofer G., et al.: Tyrosinase: a developmentally specific major determinant of peripheral dopamine. FASEB J. 2003 July; 17(10):1248-55).

Eicosanoids and Parkinson's Disease

The fact that the melanocytes of the skin do not undergo cell-death to the same extent as the dopamine producing cells of the brain in patients suffering from Parkinson's disease might depend on that the expression of prostanoids is higher in the brain than in the skin and/or that the skin continuously is renewed, which might exchange damaged cells easier that in the brain-tissue.

Prostaglandin and its analogues are members of the eicosanoid family of lipid mediators and are products of arachidonic acid metabolism. Prostaglandins are ubiquitous lipid mediators that among other functions play pivotal roles in inflammation, gene expression and cell proliferation, and in melanocytes some of these analogues have shown to have melanogenetic effects as well. The mechanism of action of these lipid messengers is thought to be primarily dependent on their interaction with specific cell surface receptors that belong to the heptahelical transmembrane spanning G-protein-coupled receptor super family. Accumulating evidence suggests that these receptors also may co-localize at the cell nucleus where they can modulate more direct gene expression through a series of biochemical events.

Cyclooxygenase type 1 and 2 (COX-1 and 2) are involved in production of prostaglandins but studies of melanocytes show that it might be primarily COX-2 that is expressed in melanocytes. The COX reaction results in the formation of the unstable endoperoxide intermediate, PGH2, which in turn is metabolized to PGD2, PGE2, PGF2α, PGI2 and TxA2. These prostanoids can be further converted to PGA2, PGB2 and PGJ2.

Eight types of membrane prostanoid receptors are expressed in humans, namely the PGD receptor (DP), four subtypes of the PGE receptor (EP1, EP2, EP3 and EP4), the PGF receptor (FP), the PGI receptor (IP) and the TxA receptor (TP). Each encoded by different genes.

The IP, DP, EP2 and EP4 receptors mediate an intracellular CAMP rise. The TP, FP and EP1 receptors induce calcium mobilization. The EP3 receptor induces a decline in intracellular CAMP. Recently it was shown that peroxisomal proliferators-activated receptors (PPARs), PPARalpha, PPARgamma and PPARdelta can bind and get activated by prostaglandins (Lim H, Dey S K. A novel pathway of prostacyclin signaling-hanging out with nuclear receptors. Endocrinology. 2002 September; 143(9):3207-10. and Lim H, Dey S K. PPAR delta functions as a prostacyclin receptor in blastocyst implantation. Trends Endocrinol Metab. 2000 May-June; 11(4):137-42.).

In brain tissue acute and chronic inflammation, interleukins and injury increase the expression of COX-2 and the release of some prostaglandins. By activating the CAMP and protein kinase A pathway, PGs enhance tetrodotoxin-resistant sodium currents, inhibit voltage-dependent potassium currents and increase voltage-dependent calcium inflow in nociceptive afferents. This decreases firing threshold, increases firing rate and induces release of excitatory amino acids, substance P, calcitonin gene-related peptide (CGRP) and nitric oxide. Conversely, glutamate, substance P and CGRP increase PG release.

In vitiligo, pigment cells get lost in areas of the skin, and several groups have shown an increased expression of CGRP and NPY (neuropeptide Y) in these white lesions and also an increased number of CGRP-positive nerve fibres in involved skin, compared with uninvolved skin and normal controls (Al'Abadie, Senior et al. 1994; Hristakieva, Lazarova et al. 2000; Liu, Bondesson et al. 1996).

NSAIDs and specific receptor- or intermediate blockers could be used to stop the harmful actions of prostanoids on brain cells and melanocytes of patients suffering from Parkinson's disease and other neurodegenerative diseases. Receptor agonists, COX-2 specific inhibitors, GRKs, arrestins and specific PKA and PKC inhibitors are examples of early blockers. More specific inhibitors in later downstream signaling could also be used to more selective inhibit the activation of a specific target gene.

Studies have proven that patients with long-term treatments with NSAIDs tend to develop Parkinson's disease to a less extent compared to the average population. It is most likely that this neuroprotective action is partly caused by the NSAIDs anti-inflammatory actions and its ability to stop the action of COXs and prostanoid production.

Transplantation Versus Other Treatment Methods

Human fetal cells from the ventral midbrain or dopaminergic neurons have already been transplanted in clinical studies in several hundred patients suffering from Parkinson's disease (for review, see Alexi T. et al., Prog Neurobiol. 2000 April; 60(5)), and a number of different cell types, including sertoli cells, adrenal cortex cells, fibroblasts, astrocytes and glomus cells from the carotid bodies, have been used in patients with Parkinson's disease and/or animal models (for review, see Alexi T. et al., Prog Neurobiol. 2000 April; 60(5)) and also the use of adenoviral-vector-DNA infected cells from the pigment epithelium of the eye in the treatment of CNS disease has been suggested (Kochanek et al., Pub. No.: US/0087859).

In Pub. No.: US 2003/0022369 A1, Fillmore et al., have transplanted terminally differentiated melanocytes in an attempt to correct the alterations in the pattern of movements in rats, caused by intracranial injection of 6-hydroxydopamine (6-OHDA) into the substantia nigra. Fillmore et al. do not provide sufficient information about the experiments regarding selection, purification and culture conditions. Thus, it is not possible to know if the changes in the rotation-pattern in the treated rats are caused by the melanocytes themselves or by contaminating cells such as fibroblasts and keratinocytes (or synergistic effects between the cells involved). The way of culturing cells referred to are not for melanocytes, but are used for the growth of bovine embryos, and culturing of oocytes and nuclear transferred cells. The source of the cells used in US 2003/0022369 is furthermore not specified.

Today there is a huge ethical resistance to use cells from brain-tissue of aborted foetuses. Also, the risk of passing on viral- and prion infections through tissues from allogeneic and xenogeneic donators remains an unsolved practical problem. Cells from other species often possess for us unknown retrovirus, integrated in their genome, with the consequence that these retroviruses get activated in an uncontrolled manner when the cells or tissue get transplanted into humans.

After a couple of tragic events where the recipient of gene modified cells have deceased as a consequence of the transplant, there is today an almost complete stop to the transplantation of gene-modified cells into humans. In gene therapy, it is difficult to know and control where the insert ends up, and if it gets inserted behind a strong promoter of an oncogene it may result in e.g. cancer for the patient. These ethical and practical problems make it even more important to use autologous non-gene modified strategies to develop treatments for the large and global patient-group suffering from Parkinson's disease.

The risks of transmission of infections, gene-modification and ethical dilemmas can be avoided by using autologous cells with the ability to produce the needed chemical substances and the important structural component such as: compartments for chemical storage; machinery for substance modification, transportation and release of transmitters and melanin; chemical delivery control; promotion of the neural protection of the local tissue etc.

As described above the pigment cells of the skin (the melanocytes) are closely related to the pigment cells of the basal parts of the brain, and their ability to produce large amounts of L-dopa as well as express tyrosinase and tyrosine hydroxylase is also well studied and described.

Melanocytes do not normally divide in the skin, but normally exist as a “chain of pearls” in a certain number at the basal-layer of the epidermis, producing their melanin that is distributed to the keratinocytes. It is only through very specific growth promoting stimuli that one can make melanocytes to divide and migrate. This argues against the risk of uncontrolled growth and migration of melanocytes after transplantation to the brain.

Nonetheless, this of course also makes the cultivating of these melanocytes in pure and undifferentiated cultures a severe task.

Interestingly, it has been described that only a few hundred thousand surviving dopamine producing cells are needed to give a clinically relevant effect after transplantation of allogeneic foetal cells. One major cause of cell-death in previous Parkinson's disease-trials using cell-therapy has been immune rejection of donor cells. Given that autologous melanocytes can be used in accordance with the present invention, the problems of rejection are likely to be eliminated.

For the first time, in accordance with the present invention, selection and culture techniques are now able to allow production of melanocytes for transplantation to patients with Parkinson's disease, which opens up for a new approach in therapy. Cultures of cells from several donors in the age-category of Parkinson's patients as well as from two patients with active Parkinson's disease have been performed in the lab of the inventors.

### SUMMARY OF THE INVENTION

The main object of the present invention is to provide human melanocytes, which are suitable for various uses, such as in implantation in patients suffering from a neurodegenerative disease, e.g. Parkinson's disease.

Said melanocytes are obtained by a melanocyte cultivation method, as disclosed by the present invention. The present cultivation method generates melanocytes which keep their normal morphology without any signs of terminal differentiation or inactivation in culture.

The cultivation method provides pure human melanocytes suitable for many uses, such as exemplified by the invention. Said melanocytes may be implanted into a patient suffering from a neurodegenerative disease, e.g. Parkinson's disease, or be used in a screening method to identify substances suitable for the development of a drug for use in the treatment of a neurodegenerative disease.

In a preferred aspect of the present invention, said melanocytes are implicated in an autologous implantation method, wherein said cells are obtained from one individual, and thereafter cultured in a preferred culture condition, followed by implantation back into the same individual, which individual is suffering from a neurodegenerative disease such as Parkinson's disease. In another equally preferred aspect, said melanocytes are monoploid melanocytes.

In another aspect of the present invention, autologous cells from an individual predispositioned to Parkinson's disease, is tested for sensitivity to certain compounds previously shown to be more likely to effect cells from a Parkinson's disease patient, by comparing the sensitivity with autologous cells obtained from a healthy individual.

The present invention predominately aims to provide a new improved treatment method for neurodegenerative diseases, such as Parkinson's disease, by using melanocytes prepared in accordance with the present invention.

### DETAILED DESCRIPTION OF THE INVENTION

Definitions

The present invention relates to a method for culturing pure undifferentiated, L-dopa and/or melanin producing human melanocytes suitable for various uses, as disclosed herein. A more detailed description of each of these uses will follow in the present section.

In the present context, a “melanocyte” is a cell which carries a pigment. The melanocyte is a cell in the basal layer of the epidermis that produces melanin under the control of the melanocyte-stimulating hormone.

An in vitro method according to the present invention generates a human cell culture which comprises at least 90% human melanocytes which are capable of melanin and/or L-dopa synthesis. Said melanocyte culture is consequently at least 90% “pure”. A “pure” human melanocyte culture in the context of the present invention, is a cell culture with a purity of at least 80 to 100%, such as 80 to 85%, 85 to 90%, 85 to 95%, 90 to 95%, 90 to 100% or 95 to 100%. Such a melanocyte culture may be at least 80, 81, 82, 83, 84, 85, 86, 87, 88, 89, 90, 91, 92, 93, 94, 95, 96, 97, 98, 99 or 100% pure. The purity of the melanocytes according to the present invention may also be lower than 80%. The purity in this context refer to the amount of melanocytes which are present in the culture.

By the term “melanin”, is meant a substance, which is produced by the melanocyte, and which substances are insoluble pigments that account for the color of e.g. skin and eye.

In the present context “L-dopa” refer to a substance with the full name L-3,4-dihydroxyphenylalanine, which is produces by e.g. a melanocyte. In the animal and human body, L-dopa is enzymatically converted to dopamine (DA), the first biologically active amine in the biosynthetic chain of tissue catecholamines.

The term “epidermal cells” is herein employed to describe cells which are derived from the epidermal cell layer, i.e. the outer layer of the skin, and which do not include cells from the dermal cell layer. Examples of epidermal cells are e.g. melanocytes and fibroblasts.

A “cell culture” is used to define a collection of more than one cell. In the present context, said cell culture is set up according to any of the cultivation methods as described by the present invention. The cells in a cell culture are allowed to divide, grow and differentiate as determined by the conditions of the cultivation method.

The term a “serum-free” medium, is a medium which is essentially free from traces of components originating from serum. Such a medium is e.g. the M2-melanocyte medium (No. C-24300, PromoCell, Heidelberg, Germany), which is free from serum, pituitary extract and phorbol esters, all to minimize the risk of carry over any viruses or prions and to minimize the risk of cell-transformation. Said medium can of cause also be a medium prepared by a user themselves, based on serum-free basal medium, specific growth factors normally expressed in the human body (such as bFGF) produced by hybrid DNA technology, wherein the DNA encoding the human growth factors is inserted in a bacteria or yeast cell and purified from there.

A “pituitary extract-free” medium is a medium which is essentially free from traces of extracts from the pituitary. An example of such a medium is the M2-melanocyte medium, which is previously disclosed herein.

A “phorbol ester-free” medium, is a medium which is essentially free from traces of phorbol esters. An example of such a medium is the M2-melanocyte medium, which is previously disclosed herein.

The term “subculturing”, which is frequently used in the context of the present invention, means that during the execution of the cultivation method, undesired cells, such as fibroblasts, keratinocytes and/or langerhan cells, are removed from the culture by various means, and the desired cells (i.e. a subculture of the original culture) are obtained and further cultured under preferred culture conditions.

“Mechanically and/or enzymatically separating”, according to the invention, means that the epidermal cells are separated from the basal cells in a skin sample, e.g. by the use of enzymes, such as any of trypsin, dispase and/or collagenase, and/or by the use of mechanical separation, such as by the shaking of a culture flask, or ultrasound vibrating of stirring the enzymatic solution for better surface contact and penetration, whichever operation is appropriate for the purpose. The object of such an operation is to obtain an essentially pure sample of epidermal cells for further use in a cell culture method in accordance with the present invention.

A “human skin biopsy” is a skin biopsy, i.e. a skin sample of any size, which is taken from a human being by any preferred method, such as by a shave-biopsy, punch biopsy, scrape sample, and/or enzymatic in vivo separation of the epidermis, which skin sample is then to be used in a method according to the present invention. In a preferred embodiment of the present invention, such a skin sample is a shave biopsy, a full-thickness skin sample, or a foreskin sample. In another embodiment, such a skin sample may be taken from any preferred source.

An “autologous” cell implantation, is an implantation procedure which takes cells from one human individual only, increases the amount of cells in a cultivation method performed under certain conditions, such as those conditions employed by the present invention, and thereafter reinserts the cells obtained back into said individual, to avoid an immune reaction often associated with the implantation of non-self material into an individual. The term “autologous” refers to the fact that the tissue is obtained from and thereafter reinserted into, the same individual.

An “implantation” in the present context, refers to the insertion of e.g. a living tissue into the body, which in the context of the present invention refers to the insertion of living melanocytes into a human being in a treatment method. The terms “implantation” and “transplantation” may be used interchangeably in the same context.

A “monoploid” human melanocyte culture, is in the present context cells that are obtained from one individual. These cells are considered to be monoploid as they comprise the same genetic material, i.e. they have identical genomes.

A “normal differentiation”, according to the present invention, is a differentiation pattern, i.e. a pattern by which the cell is changing in morphology and mission during the culturing period, which is normal for the cell being used, such as a melanocyte according to the present invention.

“Mitotic qualifications”, means that the cell in question is still qualified to divide, thus no terminal differentiation of the cell has taken place.

A “composition” according to the present invention, comprises in addition to a melanocyte obtained in accordance with a method as disclosed herein, any component which aids in forming a composition suitable for use as a medicament, and for the administration of such a medicament. Such a component may be a pharmaceutical excipient, and/or a suitable binder or a matrix, or hyaluronic acid (unsterilized or stabilized so called NASHA) or any other inert material such as silicon gel, or any other suitable means for such use.

“Screening”, according to the present invention, means that a substance of any type, such as an organic substance, polypeptide or a protein, is searched for, which substance in any way can effect the condition of the cells that are being used in such a screening method. Such screening may be performed by any preferred method, such as by a high-throughput screening method, which makes it possible to test many substances at the same time and during a short time-period. In a preferred embodiment of the present invention, screening may be performed by high-throughput screening. The substances identified by said screening method, may effect the condition of the cells used in the method, both positively and negatively. The cells which are being used in such a screening method may be both cells derived from a healthy individual, or cells obtained from a person suffering from a condition, such as Parkinson's disease.

By a “cell counter” means a technique which is used to count cells. Said technique may use a mesh grid system and a microscope to investigate the amount of viable/non-viable cells present in sample.

By “Flow cytometric analysis” is meant a tool for analyzing the structural and functional characteristics of cells or particles in suspension.

The term “fluorescence reader” is used to describe a reader which detects fluorescense emitted from a fluorescent compound of any kind. Said fluorescense reader may in the present invention be used for detecting compounds which have bound to a cell, e.g. a melanocyte, in a screening method.

The term “neuroprotective substance” is used to describe a substance which promotes the growth and viability of neural cells, or cells which possess neural cell properties, such as melanocytes.

A substance which exerts a “neurotoxic” effect on a cell, is used to describe a substance which is toxic to neural cells, or cells which possess neural cell properties, such as melanocytes.

The Cultivation Method of the Present Invention

It is a main objective of the present invention, to provide an in vitro method for generating a human cell culture which comprises at least 90% human melanocytes which are capable of melanin and/or L-Dopa synthesis. In such a method, epidermal human cells are cultured in a serum-free, pituitary extract-free and phorbol ester-free medium in the presence of antibiotics, and said epidermal cells are further subcultured in the presence of at least 0.75 mM Ca²⁺.

Such an in vitro method generating a human cell culture which comprises at least 90% human melanocytes which are capable of melanin and/or L-Dopa synthesis, may in a preferred embodiment, comprise steps to mechanically and/or enzymatically separate epidermal cells from dermal cells, thereby freeing the basal cells from the epidermis part in a skin sample, culture the separated epidermal human cells in a serum-free, pituitary extract-free and phorbol ester-free medium in the presence of antibiotics, and further subculture the epidermal cells in the presence of at least 0.75 mM Ca²⁺.

In another preferred embodiment, the present in vitro method generating a human cell culture which comprises at least 90% human melanocytes which are capable of melanin and/or L-Dopa synthesis, may comprise taking a human skin biopsy, such as a shave biopsy, a full-thickness skin sample, or a foreskin sample and mechanically and/or enzymatically separating the epidermis from the dermis, thereby freeing the basal cells from the epidermis part, and culturing the separated epidermal human cells in a serum-free, pituitary extract-free and phorbol ester-free medium in the presence of antibiotics, and further subculturing the epidermal cells in the presence of at least 0.75 mM Ca²⁺.

In another equally preferred embodiment of said in vitro method generating a human cell culture which comprises at least 90% human melanocytes which are capable of melanin and/or L-Dopa synthesis, said skin biopsy is taken from one human subject only.

The present invention also comprises an in vitro method generating a human cell culture which comprises at least 90% human melanocytes which are capable of melanin and/or L-Dopa synthesis, which is used in an autologous cell implantation, said method comprising taking a human skin biopsy, such as a shave biopsy, a full-thickness skin sample or a foreskin sample and mechanically and/or enzymatically separating the epidermal cells from the dermal cells, thereby freeing the basal cells from the epidermis part, culturing the separated epidermal human cells in a serum-free, pituitary extract-free and phorbol ester-free medium, in the presence of antibiotics, and subculturing the epidermal cells in the presence of at least 0.75 mM Ca²⁺.

The present invention also comprises an in vitro method generating a human cell culture which comprises at least 90% human melanocytes which are capable of melanin and/or L-Dopa synthesis, which is used in an autologous cell implantation, said method comprising mechanically and/or enzymatically separating the epidermal cells from the dermal cells, thereby freeing the basal cells from the epidermis part, culturing the separated epidermal human cells in a serum-free, pituitary extract-free and phorbol ester-free medium, in the presence of antibiotics, and subculturing the epidermal cells in the presence of at least 0.75 mM Ca²⁺.

Furthermore, the invention comprises an in vitro method generating a monoploid human cell culture which comprises at least 90% human melanocytes which are capable of melanin and/or L-Dopa synthesis, comprising taking a human skin biopsy, such as a shave biopsy, a full-thickness skin sample or a foreskin sample, mechanically and/or enzymatically separating the epidermal cells from the dermal cells, thereby freeing the basal cells from the epidermis part, culturing the epidermal human cells in a serum-free, pituitary extract-free and phorbol ester-free medium in the presence of antibiotics, and subculturing the epidermal cells in the presence of at least 0.75 mM Ca²⁺.

In another embodiment, the present invention comprises an in vitro method generating a monoploid human cell culture which comprises at least 90% human melanocytes which are capable of melanin and/or L-Dopa synthesis, which method comprises mechanically and/or enzymatically separating the epidermal cells from the dermal cells in a skin sample thereby freeing the basal cells from the epidermis part, culturing the epidermal human cells in a serum-free, pituitary extract-free and phorbol ester-free medium in the presence of antibiotics, and subculturing the epidermal cells in the presence of at least 0.75 mM Ca²⁺.

In a presently preferred aspect, the present invention relates to a method wherein said human melanocyte culture is at least 90% pure, such as at least 91, 92, 93, 94, 95, 96, 97, 98, 99 or 100% pure. Additionally, the present invention relates to a method, wherein said human melanocyte culture is at least 95 to 100% pure.

In another aspect, the present invention relates to a method, wherein the concentration of Ca²⁺ is at least 1 mM. Additionally, the present invention relates to a method, wherein the concentration of Ca²⁺ is at least 1 mM to 1.6 mM. The present invention also relates to a method, wherein Ca²⁺ is kept at approximately 1.2 to 1.6 mM during one or more days, of the cultivation period.

It should be emphasized, that in the method of the present invention as disclosed herein, any antibiotics which suitable for use in the present invention, which does not significantly negatively effect human melanocytes, may be used, such as G418 sulfate or gentamycin sulfate, or any antibiotics related thereto, or any other preferred antibiotics.

It should furthermore be emphasized, that the calcium concentration used during the culture method according to the present invention, may be any preferred calcium concentration of at least 0.75 to 1.6 mM such at least 1 to 1.2, 1 to 1.6, 1.2 to 1.6, 0.75 to 1.2, 0.75 to 1, 0.85 to 1 or 0.85 to 1.1. Said calcium concentration may be at least 0.81, 0.82, 0.83, 0.84, 0.85, 0.9, 0.95, 0.96, 0.97, 0.98, 0.99, 1.0, 1.1, 1.15, 1.2, 1.2, 1.25, 1.3, 1.35, 1.4, 1.45, 1.5, 1.55 or 1.6 mM. Said calcium concentration may also be kept at an elevated concentration during one or more days of the cultivation period, such as in a concentration of at least 1.2 to 1.6 mM, or any other suitable concentration.

In a preferred aspect, a method according to the present invention is used, wherein said melanocytes maintain their mitotic qualifications in the culture. The melanocytes may be kept in the culture until the desired amount of cells have been produced.

Enscoped by an aspect of the present invention is also a human melanocyte culture generated by a method as disclosed herein. Said melanocyte culture may in accordance with the present invention comprise one or more cell(s), which are autologous melanocyte(s). Enscoped by an embodiment of the present invention, are also two or more cells from a melanocyte culture, which are monoploid.

In one preferred aspect of the invention, said one or more cell(s), may be used as a medicament, said cells being obtained from a melanocyte culture, and may be monoploid and/or autologous cells.

Furthermore, the invention also comprises a composition comprising one or more cell(s) obtained from a melanocyte culture, which cells may be monoploid and/or autologus, for use as a medicament.

Transplantation of Melanocytes to Parkinson's Patients

According to a further aspect of the invention, melanocytes from culture are used to replace damaged and/or lost brain tissue in patients suffering from Parkinson's or other neurodegenerative diseases. An extra test through polymerase chain reaction (PCR) technology detecting a possible mycoplasma infection of the culture as well as other tests for detection of microbial contaminations should be necessary before the transplantation to brain-tissue is to occur

At the transplantation of autologous melanocytes, the melanocytes are enzymatically released from their culture chambers, washed twice in low-calcium medium, spun down to a pellet and resuspended in a very small volume of nutritious solution, and thereafter through a micropipette distributed to the area where the non-pigmented epidermis have been peeled off.

Biopsies can be taken at a hospital far away from the laboratory and transported in test-tubes containing a low calcium, low magnesium medium. In right transport conditions the pigment cells in the biopsies will stay viable in the tube for over 48 hours, before being released from the tissue and put into cell-culture.

At the delivery of skin melanocytes into Parkinson-patient's brain a small volume washed and cleaned cells can be transferred with help of stereotactic technology into the brain through small holes in the cranium as has already been practised in several hundred patients treated with dopamine-producing cells from aborted foetuses.

Melanocytes have been successfully frozen over a period of one year and then thawed and transplanted to white skin lesions with good re-pigmentation and permanent results seen at long-term follow-ups (Olsson M J, Moellmann G, Lerner A B, Juhlin L: Vitiligo: Repigmentation with cultured melanocytes after cryostorage. Acta Derm Venereol (Stockh) 1994; 74: 226-228.). This ability for the melanocytes to survive through long-distance transportations and that it is possible to freeze and thaw cultured cells without any major loss of cell-numbers or specific functions, makes it possible for a highly-specialised laboratory to deliver and support the need of cells for a large geographical area.

Autologous cells are preferably used, but if the melanocytes from the Parkinson patient to be transplanted are fragile and very vulnerable to chemical stress, allogen or dopamine producing stem cells could be used.

Melanocytes can easily be taken from the patient to be treated (autologous), through a superficial non-harmful shave biopsy under local anaesthesia and cultured through selection where irrelevant cell-types are eliminated. Highly selective melanocyte-promoting medium called M2 (PromoCell, Heidelberg, Germany, Catalogue no.: C-24300, www.promocell.com/en/pdf/C-24300.pdf) can be used and short-term incubation of G418 eliminates the contamination of fibroblasts and short-term elevation of the Ca²⁺ level differentiates contaminating keratinocytes.

When subculturing, the remaining weakened fibroblasts and differentiated keratinocytes will not stick to the culture flasks and they will get lost next time the medium is changed. The culturing process occurs during a couple of weeks and at the end of that time there are several millions of pure melanocytes ready to be transplanted or used for other studies.

Accordingly, the present invention relates to the use of one or more cell(s) obtained from a melanocyte culture as disclosed herein, for the preparation of a pharmaceutical composition for use in an autologous cell implantation. Said cells may be monoploid and/or autologous.

Furthermore, the present invention also relates to the use of one or more cell(s) derived from a melanocyte culture as disclosed herein, for the preparation of a pharmaceutical composition for the treatment of Parkinson's disease in a patient in need thereof. Said cells may be monoploid and/or autologous.

In another preferred aspect, the present invention relates to the use of one or more cell(s) derived from a melanocyte culture as disclosed herein, for an autologous cell implantation for the treatment of Parkinson's disease in a patient in need thereof. Said cells may be monoploid and/or autologous.

In yet another preferred aspect, the invention relates to a method for treating a human patient suffering from Parkinson's disease, comprising implanting one or more cell(s) from a melanocyte culture derived from a melanocyte culture as disclosed herein, into said patients striatum and/or substantia nigra. Additionally, the invention relates to a method for treating a human patient suffering from Parkinson's disease, comprising implanting one or more cell(s) which are autologous or monoploid, into said patients striatum and/or substantia nigra.

Screening of Neurotoxic and Neuroprotective Substances

According to one aspect of the present invention, melanocytes and in particular melanocytes from Parkinson patients, are used in screening of substances, which can act as new neuroprotectants or as neurotoxins. According to a further aspect the invention covers the use of such substances for treatment.

Cells from the CNS are difficult to culture and the access to viable adult human tissue is very limited, which makes it even more important to create functional systems based on cells that are easily available, not harmful for the donors and give rise to less ethical resistances and issues.

What makes the melanocyte suitable as a screening model is its close relation to other nerve cells, as well as the possibility to use completely defined cell-culturing system free from pituitary extracts and serum as well as to culture large amounts of these cells under fully controlled conditions.

It is possible to in a reasonable short time using Geniticin (G418 to selectively suppress the fibroblasts) and Ca²⁺ (differentiate and eliminates the keratinocytes) incubation steps to get enough quantities of at least 90%, such as at least 95, 99 or 100% percent pure melanocyte cultures for high throughput screening (HTS) of larger compound library in 96 or 384 wells format.

It is also possible, in accordance with the present invention, to use any other antibiotics which do not effect melanocytes, such as gentamycin sulfate, or any other preferred antibiotics. It should also be acknowledged, that the calcium concentration of the method may be varied between any of the concentrations, as disclosed herein.

Melanocytes can be plated in substance pre-plated plates. This can be done either by hand with a multipipette in the 96 wells (or less) format or preferable in a robotic system that takes care of all plating steps in both 96 and 384 wells format. After a decided incubation time (2 h-3 days), the plates can be red either on a fluorescence-reader of a β-counter (scintillator), depending on the markers used. The plate used in the context of the present invention may be any plate suitable for such a method, such as a plastic plate.

Cells can be studied upon (but not limited to) DNA-synthesis, mitogenicity, cell-death and apoptosis. Different specific markers to study caspase activity, DNA-fragmentation and mitochondria membrane potential can be used to characterise apoptosis. Toxic and lethal dosages of different substances can easily be revealed and compared between Parkinson's donors and age matching healthy controls.

Accordingly, the present invention relates to a method for screening for substances capable of effecting neuronal cells from a human patient suffering from Parkinson's disease, which method comprises generating a more than 90% pure autologous human melanocyte culture from said patient, pre-plating a plate with one or more potentially effective substances, plating one or more cell(s) from said melanocyte culture onto said plate, incubating said melanocytes with said substances during a decided incubation time, and analysing the plates to identify the substances that display an effect on the plated cell(s).

In another embodiment, the invention relates to a method for screening for substances capable of effecting neuronal cells from a human patient suffering from Parkinson's disease, which method comprises employing a more than 90% pure autologous human melanocyte culture from said patient, pre-plating a plate with one or more potentially effective substances, plating one or more cell(s) from said melanocyte culture onto said plate, incubating said melanocytes with said substances during a decided incubation time, and analysing the plates to identify the substances that display an effect on the plated cell(s).

The term “substances capable of effecting neuronal cells”, refer to substances which may effect the melanocytes used in the method of the invention, either positively or negatively by e.g. suppressing growth, stimulating growth, induce proliferate, induce differentiation, induce transformation etc. Such substances identified by said method may be used for a purpose suitable for the effect of said substance.

In yet another embodiment, the present invention relates to a screening method as disclosed herein, wherein said melanocytes are at least 90% pure, such as at least 91, 92, 93, 94, 95, 96, 97, 98, 99 or 100% pure. Additionally, the present invention relates to a screening method as disclosed herein, wherein said melanocytes are at least 95 to 100% pure.

Furthermore, the present invention relates to a screening method as disclosed herein, which is executed as a high-throughput screening. The present invention also relates to a screening method as disclosed herein, wherein the plates are analysed on a reader such as a fluorescence-reader.

In yet another preferred embodiment, the present invention relates to a screening method as disclosed herein, wherein the non-viable cell(s) effected by a substance from the plate are removed, and the viable cells are analysed using a cell counter or flow cytometric analysis.

The present invention also relates to a screening method as disclosed herein, wherein said melanocyte(s) used in said method is/are generated by a method as disclosed herein.

In yet another preferred embodiment, the invention relates to the use of a screening method as disclosed herein, for screening for substances being lethal to melanocytes from patient's suffering from Parkinson's disease.

In yet another equally preferred embodiment, the invention related to the use of a screening method as disclosed herein, for screening for neuroprotective substances effective for treating Parkinson's disease in said human patient.

Furthermore, the invention relates to the use of a screening method as disclosed herein, for screening for substances that have a neurotoxic effect in a human patient.

The invention also relates to the use of a screening method as disclosed herein, for identifying a substance suitable in an individual medical treatment method for a human patient suffering from Parkinson's disease. Such an individual treatment method may be any method which is suitable for the particular patient in need. Said patient may undergo medical examinations to decide upon such a treatment.

There is also an object of the present invention to provide a substance identified by a screening method as disclosed herein. Such a substance may be used as a medicament. Such a substance may also be used in the manufacture of a medicament for the treatment of neurodegenerative diseases, such as Parkinson's disease.

Furthermore, the invention relates to the use of such a substance for treating Parkinson's disease in a human patient.

Screening for a Disposition for Parkinson's Disease

By screening a small library of chemical compounds, the present inventors have found that melanocytes from Parkinson patients (donators with Parkinson's disease) have been more vulnerable to analogues of certain substances in comparison to melanocytes derived from healthy control persons.

Therefore, in a preferred embodiment of the invention, cells from patients are used. The receptor-mediated signal transduction, which these substance analogues in the family of prostanoids (but not restricted to this family of chemical substances) are mediating, is known and there are possibilities to inhibit specific steps in these cascades. Novel neuroprotective drugs can be detected through this kind of screening that can be of valuable use to e.g. make long-term cultures of neurons possible.

Such screening and substances identified from such screening can also be used to identify lead compounds or drugs to develop medicines to treat Parkinson's disease or other diseases related to increased neuronal death with or without a higher susceptibility to neurotoxins or other endogenous or exogenous substances and compounds that may act harmful or beneficial for the survival and growth of the cells. With this method identified neuroprotective substances can also be useful in medication of Parkinson's patients that have undergone a dopa-producing cell-transplantation, and hereby enhance the chances for the implanted cells (or tissue) to survive.

The usefulness of screening to find damaging or protective substances especially for Parkinson's disease is based on the finding that the same inborn vulnerability in the pigment cells in the substantia nigra and striatum is also present in the pigment cells of the skin, which are indeed very close related to the pigment cells in substantia nigra and striatum. This is underscored by further findings showing drastically increased sensitivity in melanocytes from Parkinson's donators (donators with Parkinson's disease) in the presence of certain substances compared to melanocytes from control individuals. This can also result in an understanding of the cause of Parkinson's disease and the benefits to develop drugs that inhibit these mechanisms can hardly need more emphasis.

Accordingly, the present invention also relates to a method for screening for a predisposition for Parkinson's disease in a human patient, comprising testing the sensitivity of one or more cell(s) from an autologous melanocyte culture obtained from said patient for a substance identified by a screening method as disclosed herein.

In another aspect, the present invention also relates to a method for screening for a predisposition for Parkinson's disease in a human patient, which comprises a screening method as disclosed herein.

In another preferred aspect, the present invention relates to a method for screening for a predisposition for Parkinson's disease in a human patient, comprising testing the sensitivity of one or more cell(s) from an autologous melanocyte culture from a patient predisponary for Parkinson's disease for a test substance, and comparing the sensitivity of said patient's one or more cell(s) from an autologous melanocyte culture, to the sensitivity of one or more cell(s) from an autologous melanocyte culture from a healthy individual.

Furthermore, the invention relates to a method, wherein the sensitivity of said cells from two different autologous melanocyte cultures is tested using a screening method comprising generating a more than 90% pure autologous human melanocyte culture from each patient/individual, pre-plating a double set of plates with identical one or more potentially neurotoxic substance(s), plating one or more cell(s) from one of said melanocyte cultures onto each set of plates, incubating said melanocytes with said substances during a decided incubation time, and analysing the plates to identify the effect that the substances have on the plated cell(s), comparing the sensitivity of the melanocyte cultures from each patient/individual to the substances.

The invention also relates to a method for screening for substances capable of effecting neuronal cells from a human patient suffering from Parkinson's disease, which method comprises employing a more than 90% pure autologous human melanocyte culture from each patient, pre-plating a double set of plates with identical one or more potentially neurotoxic substance(s), plating one or more cell(s) from one of said melanocyte cultures generated in step a) onto each set of plates, incubating said melanocytes with said substances during a decided incubation time, and analysing the plates to identify the effect that the substances have on the plated cell(s), comparing the sensitivity of the melanocyte cultures from each patient to the substances.

In another aspect, the present invention relates to a method, wherein said melanocytes are at least 90% pure, such as at least 91, 92, 93, 94, 95, 96, 97, 98, 99 or 100% pure. Additionally, the invention relates to a method, wherein said melanocytes are at least 95 to 100% pure.

Furthermore, the invention relates to a method, wherein the plates are analysed on a reader, such as a fluorescence-reader. The invention also relates to a method, wherein the non-viable cell(s) effected by a substance from the plate in step e), are removed, and the viable cells are analysed using a cell counter or flow cytometric analysis.

In a preferred aspect, the invention relates to a method, wherein said melanocyte(s) used in said method is/are generated by a method as disclosed herein.

Substances that Act Harmful and Lethal to Melanocytes from Parkinson's Patients

In a screening of a substance library, the present inventors have found that a category of substances belonging to the family prostanoids, have a harmful effects on Parkinson's melanocytes.

There are especially some of these prostanoid analogues belonging to the subgroup prostaglandins that have a very potent effect on melanocytes derived from Parkinson's donors. The specific prostaglandins acts as ligands and bind to specific 7-TMS G-protein receptor, which have their specific signal transduction pathway that is lethal for the Parkinson's melanocytes. Receptors specific for these prostaglandin analogues can also be expressed at the nucleus and in that way act more direct on the gene control that also may contribute to the harmful mechanisms involved. Reports that patients on long-term anti-inflammatory medication are less prone to develop Parkinson's disease support the present findings.

Accordingly, the present invention also relates to the use of a screening method as disclosed herein, for screening for substances being lethal to melanocytes from patient's suffering from Parkinson's disease.

Furthermore, the present invention also relates to the use of substances shown to be specifically harmful to cells obtained from Parkinson's patients. Such substances, e.g. the prostaglandins PGF2a, PGE2, PGA2 and PGJ2, may be used in long-term exposure studies, to investigate e.g. the presence of a predisposition for Parkinson's disease in a human patient. Such substances may also be used in an assay to test for sensitivity, in according with the invention. Consequently, such substances may be used in any screening method according to the invention.

The difference in vulnerability to certain substances between Parkinson's versus normal melanocytes can easily be used when setting up HTS (High-Throughput Screening) in the search for factors that are lethal for Parkinson's pigment cells. LD50 levels etc could easily be pinpointed and verified and the group of substances involved gives a clue of the lethal mechanisms of actions involved.

Melanocytes as Supporting Cells

It has been shown that retinal pigment epithelial cells (RPE) produce a localized immunosuppressive environment at the site of transplantation (Allen R., Use of pigmented retinal epithelial cells for creation of an immune privilege site. Intern. Publication no. WO 99/34834). The local immunosuppression is caused by the secretion of Fas-L protein. Fas-L expression kill activated lymphocytes that invade the site of transplantation. It is most likely that the closely related melanocytes also express Fas-L, which would make an allogen or xenogen transplantation of melanocytes to the recipients brain less vulnerable to immunological rejection than many other types of cells not expressing Fas-L protein would face.

The likely expression of Fas-L protein by human skin and hair melanocytes can also be useful in transplantation of other non-autologous cells to a recipient. The Fas-L expressing melanocytes would be co-transplanted with another therapeutic cell and create a local immunosuppressive environment that would protect the transplanted cells or tissue from immunological attack and rejection. The melanocytes used to facilitate the survival of the grafted cells or tissue could be of a non autologous or most preferable of an autologous origin.

As example but not limited by the examples below, autologous melanocytes could be co-transplanted and used as locally supporting cells in the transplantation of Langerhan's islets, insulin producing beta-cells, bon-morrow cells, neural cells to the brain tissue and to support and protect specialised or genetically transformed stem-cells. Melanocytes might also be suitable as carrier of therapeutic genetical material in treatments of various disorders of the skin or brain, where an important protein is missing or non-functioning due to mutation. Depending on if the donor melanocytes are of autologous origin or from allogen left-over tissue derived from skin-reduction surgery the process of preparing the cells are slightly different.

Accordingly, the present invention also relates to the use of melanocytes according to the present invention, as supporting cells in a non-autologous or autologous transplantation

Other Culturing Methods and Co-Cultivation with Neural Tissue and Free Neural Cells

Melanocytes have under the right culture conditions active anchor proteins, and stretch out their connecting dendrites, which makes co-cultivation with other relevant cell types, e.g. glial cells and neural tissue possible. Therefore, the present invention also relates to the use of melanocytes obtained by a method according to the present invention, In a co-cultivation method.

The development of such optimal culture conditions, suitable for these purposes, have been of key importance. To reach this goal, several years of screening to find substances and combinations of substances able to affect the melanocyte have been performed. Today melanocytes from donators up to the age of 70 years both normal and Parkinson derived has been successfully cultured.

Like neural pigment cells, melanocytes produce L-dopa in large quantities but to convert this to dopamine, L dopa decarboxylase (DDK) is needed. The present inventors have still not studied the expression of DDK-mRNA or DDK-protein synthesis in melanocytes, but it is well known that DDK is expressed in neural tissues from basal parts of the brain.

Melanin-containing cells normally act detoxifying, antioxidative and buffering for their immediate environment. Melanins are polyanions, and substances with cation properties, such as amines and metal ions are easily bound to melanin by ionic interaction (Mårs, U.: Melanogenesis as the Basis for Melanoma Target. In: Dept of Pharmaceutical Biosciences, Division of Toxicology. Uppsala University, Uppsala, 1998. ISBN 91-554-4246-3)

In the skin, the pigment-producing cells also have such protective functions and the eumelanin is proved to function as a buffer, entrapping metal ions and quench or scavenge photochemically generated free radicals (Jimbow, K.: Current update and trends in melanin pigmentation and melanin biology. Kelo J Med 1995, 44, 9-18).

Also neuronal pigment cells have the ability to store and detoxify some substances in a diligent manner. Tyrosinase has shown to have a protective effect on dopamine cytotoxicity through the enzyme's ability to convert dopamine to dopaminequinone through oxidation, which in turn can be converted further to neuromelanin (J. Matsunaga et al.).

What kind of enzymes that will get upregulated in the melanocytes in the presence of cells or tissue from the central nervous system (CNS) or periphery nervous system (PNS) and how the melanocytes will influence the neural cells is not yet fully understood, but this co-cultivation might change the expression pattern in a useful way. However, to develop cultures of melanocytes in the presence of nerve cells, or to study cultured melanocytes on sections from brain tissue is by the present culturing technique possible.

In these culture chambers the level of dopamine and other transmitters/molecules can easily be detected and measured, with or without the addition of other conditioning substances, and furthermore, changes in levels of dopamine (and other transmitters), expression of related enzymes or activity of other downstream mediators, responsible for formation of neuro-melanin and cell signal transmission, can in a controlled manner be defined and quantified. Growth factor stimulation could be used for induction of dopa-decarboxylase or other protein expressions.

One can also gain knowledge about nerve cells in culture and their ability to survive longer in the presence of melanocytes, which can act antioxidatively or detoxifying (see above).

One can also use melanocytes as a “feeder-layer/synergetic enhancers” making it possible for nerve cells, which are today not possible or difficult to culture, to grow in close contact to melanocytes and therefore being possible to study in culture.

Furthermore melanocytes have been shown to be able to function as antigen-presenting-cells and could therefore be of benefit in immuno-studies of other cell types prone to be attacked by the immune-system.

Co-cultivation between muscle cells and nerve cells has been proven to be useful in micro-studies of the muscle contraction (WO 03/040300 A2) and different cell types have been suggested to be combined in different ways and cultured separated by nylon and polycarbonate membrane barriers. The study of the cells separated in different chambers would allow the studies of for example medium release of excreting factors such as substance-P, cytokines, growth factors and antibodies (WO 02/074902 A2).

In order to study real interaction and direct compound exchange between the interacting cells co-cultures with direct contact seems to be the only way. Further this culture technique makes it possible to culture and study neurons, difficult to be cultured on their own. Melanocytes would help supporting the co-cultured cells (e.g. neuron) allow them to stick to a surface and direct exchange growth promoting factors through efficient touch-exchange.

Further the co-cultures would be useful in studies of neurotransmission, neuroprotection, neurodegeneration and cultivation of neurons to be used in transplantation treatment to correct neurotissue damage after stroke or spinal cord injury (to fill out the gap). If the co-cultured melanocytes are of autologous origin it might not hurt if they follow the transplant to support their cells in their new environment. If allogeneic or xenogeneic melanocytes are used in the co-cultures, the different cell types can easily be separated by gradient centrifugation with Fillcoll or Percoll (Amersham Pharmacia Biotech, Uppsala, Sweden) prior transplantation into human brain or spinal cord.

The present invention is further exemplified in the following experimental section. It should however be emphasized, that the invention is not limited to any of the conditions as specified in the below.

Experimental Section

Cultivation Method

Donor Tissue

From Shave Biopsies:

A normally pigmented area of about 2×4 cm² in the gluteal region was anaesthetized with a solution containing equal amounts of 10 mg/ml lidocaine and Tribonat® (bicarbonate solution from Fresenius Kabi, Uppsala, SWEDEN). A superficial shave biopsy (as thin as possible) was taken with a Goulian-Weck skin graft knife (Edward Weck & Company, Inc, Research Triangle Park, N.C.). The Goulian knife should be equipped with a 006 shield, to support very shallow biopsies. The specimens were put in a 15 ml test-tube containing S-MEM, i.e. Joklik's modified minimal essential medium (GIBCO BRL, Life Technology, Gaitersburg, Md.) and transferred to the laboratory for preparation. The donor area was covered for a week with Tegaderm™ (3M, St Paul, Minn.).

In cases with transportations between 1 h to 4 h, the medium is furnished with antibiotics and kept at 8° C. At transportations over 4 h the biopsies are put in complete M2 medium (cat. No. C-24300, PromoCell) and kept at 8° C.

From Full-Thickness Skin Samples:

Left-over skin from surgical reconstruction such as breast and abdominal reduction-surgery can be used to prepare melanocyte-cultures from. 50 ml tubes or larger containing low-calcium and low-magnesium medium such as S-MEM with 50 U/ml penicillin and 0.05 mg/ml streptomycin is given to the surgical department and stored in refrigerator until use. At surgery the left-over skin is put in the media-containing tubes and transported to the laboratory. If you will not have the possibility to take care of the tissue at once it will stay fresh refrigerated in the tube for 30 hours. At preparation, begin with putting and stretching out the whole tissue in a large petri-dish containing 70% ethanol and let soak for about 30 seconds (to eliminate possible microorganisms). Then rinse twice in 10° C. PBS-solution. Turn the epidermal side downwards facing the bottom of the petri-dish and fill the dish to the half with the PBS-solution. Cut through the fat-tissue and the main part of the dermis with a sharp eye-scissors. Make the remaining skin sample as thin as possible, without cutting to many holes through it and remove the cut-away fat and dermal fragments and change PBS as often as necessary. It is important that the sheet of skin get thin so that the enzymes in the next step can do their job in a satisfactory way.

From Foreskin Samples:

Foreskin can be collected from the nursery in test-tubes containing S-MEM with 50 U/ml penicillin and 0.05 mg/ml streptomycin. The tubes are kept in refrigerator until preparation of cells. The pieces of skin will stay fresh for 50 hours.

The little foreskin piece is dipped in 70% ethanol twice and then washed twice in PBS to ensure that possible microbes will be eliminated. The ring structure are cut-open and stretched out in a Petri-dish with the dermis side upwards. The dermis is removed as much as possible by flat-cutting with a small eye-scissors. Make the remaining skin sample as thin as possible, without cutting to many holes through it and remove the cut-away dermal fragments and change PBS as often as necessary.

Release and Preparation of Cells

The thin donor sample is in a laminar flow-hood put into φ 6 cm or φ 10 cm Petri dish and washed once with 4 or 8 ml 0.20% w/v trypsin and 0.08% w/v EDTA in 80% v/v phosphate-buffered saline (PBS) (all SVA, Uppsala, Sweden) and 20% v/v Joklik's modified minimal essential medium and refurnished with 5 or 10 ml of the above trypsin/EDTA solution. The sample is turned back and forth with the help of jeweler's forceps to ensure that it comes in complete contact with the solution, and finally, with the epidermis side upwards, torn into pieces of 4 cm2. Make sure that there are no air-bubbles under the thin fragments. The Petri dish is incubated at 37° C. in 5% CO₂ for about 50 minutes for thin shaves, about 2-3 hours for thin-cut biopsies deriving from full-thickness skin samples and about 2 hours for thin-cut foreskin. After about half incubation time the pieces are moved around and pressed on a little, with a curved forceps, to ensure that the whole tissue will get soaked in with the trypsin/EDTA-solution.

If the delivery of the full-thickness or foreskin specimen is in the late afternoon it can after the above thin-cutting procedure be incubated in the above trypsin/EDTA solution at 4-8° C. over night and next morning if necessary incubated in 37° C. for about 1 hour or until epidermis can be removed from the dermal part (superficial shave biopsies shall not be incubated over night in trypsin/EDTA).

After incubation, the trypsin/EDTA solution is removed and about 3 ml (15° C.) trypsin inhibitor 0.5 mg/ml (Sigma, St Louis, Mo.), in PBS, is added to the dish, to discontinue the trypsin reaction. The epidermis was removed from the dermis with help of forceps and the dermis is transferred to a test-tube containing about 5 ml of the highly balanced, serum-free, melanocyte medium M2 (PromoCell, Heidelberg, Germany, cat. no. C-24300) and vortex-mixed for 5 s. The dermal pieces were then fished up with the tip of a Pasteur pipette or with the help of a hooked forceps and then discarded. The epidermal pieces were scraped with a curved jeweler forceps so that all basal cells gets free and then minced to smaller fragments and transferred, together with the trypsin inhibitor, to a test-tube. The tube was vortex-mixed for 30 s. The Petri dish was rinsed twice with a small volume of sMEM, which was also added to the tube, and then centrifuged for 7 min at 190 g (sometimes it can be difficult to pellet the cells due to free DNA and collagen which forms mucous-like structures. In these cases the content in the test-tube should be run up end down a few times in a Pasteur pipette with sharp glass-edge, which will cut down the structures to shorter fragments which facilitate for the cells to be spun-down to a pellet).

The supernatant and the floating stratum corneum-granulosum fragments are removed and the pellet is resuspended in 5 ml M2 melanocyte medium and transferred to a 75 cm2 or 150 cm2 culture flask containing M2 medium for culturing. The empty test-tube is rinsed once with 1 ml M2 which is transferred to the culture flask, to ensure that all cells get collected. The culture flask is lying flat when adding the cell-suspension, so the cells not will stick to the sides or ceiling of the flask. This is to ensure a maximum exchange rate.

A 75 cm² flask should have a total of about 15 ml medium and a 150 cm² flask should have about 30 ml M2 medium.

Culturing of Melanocytes

The M2-melanocyte medium (No. C-24300, PromoCell, Heidelberg, Germany) is free from serum, pituitary extract and phorbol esters, all to minimize the risk of carry over any viruses or prions and to minimize the risk of cell-transformation. The growth-promotion enhanced by M2-medium is brought forth by human growth factors produced by hybrid-DNA technology; there the human code for the growth factor has been inserted in the genome of yeast cells. All growth factors used are naturally according in the human skin.

The medium in the culture-flasks should be changed every third day and after 10 days it can be supplemented with 45 μg/ml geniticin for 3 days in order to selectively suppress the fibroblasts. If the geniticin incubation is abandoned the incorporation of fibroblasts will still only become about 5-10%, when culturing with M2-medium. The cells are lifted and subcultured when the culture becomes confluent. When subculturing we will get rid of most of the remaining keratinocytes since the Ca²⁺ level is about 1 mM and in that concentration most of the keratinocytes will differentiate and stop dividing and loose there ability to stick to a new culture-flask when subcultured. If this keratinocytes differentiation is desired to be enhanced, the level of Ca²⁺ could be elevated to 1.6 mM for two days.

After about 2 weeks in culture the number of cells from one biopsy varies from 10×10⁶ to 50×10⁶ (depending on the size of the sample and the age of the donor) and are then ready to be used for transplantation or other studies.

Immediate before transplantation the cells are freed from their culture flasks. Medium is sucked off and about 5 ml 37° C. trypsin/EDTA solution is added to each 150 cm2 flask. The flask is tilted back and forth a few times to ensure that the solution get in contact with all cells and then incubated in 37° C. for about two minutes. After the incubation you hit the flask with the palm of the hand on one side at the same time as you are holding the flask with the other hand. This gives the flask a jerking acceleration sideways releasing the cells from the surface. Quickly pipette over the free cells to a 15 ml test-tube containing 4 ml room tempered trypsin inhibitor (Soy-bean extract from Sigma, St Louis, Mo. in PBS). Wash out the remaining cells from the flask with help of additional 5 ml room tempered trypsin inhibitor and pipette it over to the same test-tube. Gentle spin the cells down to a pellet (about 180 g for about 6 minutes). Suck of the supernatant and resuspend (wash) the cells in 6 ml room tempered s-MEM medium without any additives. Centrifuge the cells for about 4 minutes at 180 g, suck of the supernatant and the cells are ready to be resuspended in an extremely small volume of S-MEM or PBS to be used for transplantation; or to be resuspended in desired media condition and volume to be used in experiments of interest.

Cryopreservation

Left-over cells or cell you wish to store or ship very far are lifted with the help of the mentioned trypsin/EDTA-solution, centrifuged into a pellet and resuspended in 1 ml cryoprotectant for each 10 million cells. The cryoprotectant solution consisted of 8% dimethyl sulphoxide, DMSO (Mallinckrodt, Inc., Paris, Ky.) In undiluted newborn-calf serum or of any of on the market suitable cryoprotectants. Cells and protectant are mixed by gentle pipetting and transferred to a 1.8 ml cryotube/ml. The tubes are put on ice for 10 minutes to allow the DMSO to penetrate the cells and then placed in −70° C. to −85° C. for storage or cells can direct after transferring into the cryotube be put straight into a room-tempered NALGENE freezing camber (cat. No. 5100-0001) to achieve a −1° C./min rate of cooling.

For long-term storage, move over the frozen tubes after 24 h from the −80° C. to a −150° C. freezer or to a chamber of liquid nitrogen immersion storage.

Defrosting

After storage in frozen condition, the cells are defrosted by placing the cryotube into a 37° C. water bath. Immediately after the contents are thawed (1-2 min), the cell-containing solution is carefully transferred (cells are fragile immediate after they have been frozen) to a test-tube containing culture medium and centrifuged at 150 g for 4 minutes. The supernatant is removed by pipette and the cell-pellet carefully resuspended in culture medium, and transferred to a culture flask for continues cultivation or carefully washed in sMEM once more and centrifuged and ready to be used for transplantation. The survival rate of cells that have been frozen for one year is about 70% (Olsson et al., Acta Derm Venereol (Stockh) 1994; 74: 226-228.).

### SUMMARY

Summarising the proposed technology, biopsies from Parkinson-donators can be put in nutritive solution and delivered to a suitable production-facility at almost any distance from the donor, prepared and cultured, be frozen in cryo-tubes and shipped back to the donator's hospital where the cells can be thawed, cleaned and then transplanted. And melanocytes from Parkinson's donors can be used in HTS screening in the search after substances lethal or protective to the cells, and hereby find strategies in the future medical treatments of Parkinson's disease.

Screening for Substances

Method and Results

In screening of cultured and purified melanocytes from two Parkinson's donors and controls from several healthy adult donors it was found that some prostaglandin analogues or metabolites to prostaglandin are lethal in a concentration range of 1/100 to 1/10 of what was found harmful for the control melanocytes.

The Parkinson's derived melanocytes vulnerability was strikingly noticeable in contact with PGF2a, PGE2, PGA2 and PGJ2. In short time exposures (24 h) in concentration ranges between 0.5 μg/ml and 5.0 μg/ml where analyze of the DNA syntheses was detected with 3HdT incorporation and scintillation counts no significant different in vulnerability between Parkinson's melanocytes and normal adult control melanocytes could be established. In long-term exposure (10 days) to the above mentioned prostaglandins, only two substances, namely PGJ2 and PGA2 had a negative impact on the growth of the control melanocytes and only at the highest concentration step 5.0 μg/ml and no specific cell death was noticeable. But the 10 days exposure of the Parkinson's melanocytes to the prostaglandins, resulted in a complete cell-death within 8 days in culture at the whole concentration interval (0.5-5.0 μg/ml) for all substances studied. The Parkinson's melanocytes in the negative control and positive control wells (at the Parkinson's plate) were viable and no cell death was visible, pointing out that the cell death was related to the long-term exposure to the prostaglandins.

These findings suggests that there is an inborn vulnerability (sensitivity) to certain substances that is shared between the cells in the brain that are damaged and/or lost in the Parkinson's disease and the melanocytes of the skin in patients suffering from Parkinson's disease. These cell-types are closely related in the line of development.

Future Experiments

Using animal models of Parkinson's disease e.g. the 1-methyl-4-phenyl-1,2,3,4-tetrahydropyridine (MPTP) or 6-OHDA models (different types of chemically induced Parkinsonism like symptoms caused by cytotoxicity) and stereotactic frames for controlled local application of the cells, will be used to establish the most suitable delivery method of pure human melanocytes. MPTP-apes could be used to get a model closer to humans in terms of practical transplantation technicalities and result-evaluation, than what the rats can offer.

### REFERENCES

Hara M et al, Innervation of melanocytes in human skin. J Exp Med. 1996 Oct. 1; 184(4):1385-95
Lerner M R, Tools for investigating functional interactions between ligands and G-protein-coupled receptors. Trends Neurosci. 1994 April; 17(4):142-6).
Marles L. K. et al, Regulation of Tyrosinase and tyrosine hydroxylase activities by α- and β-MSH. Pigment Cell Res. Vol 15, supplement 9, 2002, PO-010, page 59)
Mårs, U., Melanogenesis as the Basis for Melanoma Target. In: Dept of Pharmaceutical Biosciences, Division of Toxicology. Uppsala University, Uppsala, 1998. ISBN 91-554-4246-3).
Jimbow, K., Current update and trends in melanin pigmentation and melanin biology. Kelo J Med 1995, 44, 9-18
Eisenhofer G., et al., Tyrosinase: a developmentally specific major determinant of peripheral dopamine. FASEB J. 2003 July; 17(10):1248-55
Lim H, Dey S K., A novel pathway of prostacyclin signaling-hanging out with nuclear receptors. Endocrinology. 2002 September; 143(9):3207-10.
Lim H, Dey S K., PPAR delta functions as a prostacyclin receptor in blastocyst implantation. Trends Endocrinol Metab. 2000 May-June; 11(4):137-42.
Alexi T. et al., Prog Neurobiol. 2000 April; 60(5)
Olsson M J and Juhlin L., Long-term follow-up of leucoderma patients treated with transplants of autologous cultured melanocytes, ultra-thin epidermal sheets and basal cell-layer suspension. Br J of Dermatology 2002; 147: 893-904
Olsson M J, Moellmann G, Lerner A B, Juhlin L., Vitiligo: Repigmentation with cultured melanocytes after cryostorage. Acta Derm Venereol (Stockh) 1994; 74: 226-228.
Allen R., Use of pigmented retinal epithelial cells for creation of an immune privilege site. Intern. Publication no. WO 99/34834
Olsson et al., Acta Derm Venereol (Stockh) 1994; 74: 226-228.

1-35. (canceled)

36. An in vitro method for generating a human cell culture which comprises at least 90% human melanocytes which are capable of melanin and/or L-dopa synthesis, comprising
a) culturing epidermal human cells in a serum-free, pituitary extract free and phorbol ester-free medium in the presence of antibiotics, and
b) subculturing the epidermal cells in the presence of at least 0.75 mM Ca²⁺.

37. An in vitro method according to claim 36, wherein step a) is preceded by the step of mechanically and/or enzymatically separating epidermal cells from dermal cells.

38. An in vitro method according to claim 36, for use in an autologous cell implantation.

39. An in vitro method according to claim 36, for generating a monoploid human cell culture.

40. A method according to claim 36, wherein said human melanocyte culture is at least 90% pure, such as at least 91, 92, 93, 94, 95, 96, 97, 98, 99 or 100% pure.

41. A method according to claim 36, wherein said human melanocyte culture is at least 95 to 100% pure.

42. A method according to claim 36, wherein the concentration of Ca²⁺ is at least 1 mM.

43. A method according to claim 36, wherein the concentration of Ca²⁺ is at least 1 to 1.6 mM.

44. A method according to claim 36, wherein the concentration of Ca²⁺ in the culture is kept at approximately 1.2 to 1.6 mM during one or more days of the cultivation period.

45. A method according to claim 36, wherein said melanocytes maintain their mitotic qualifications in the culture.

46. A human melanocyte culture generated by a method according to claim 36.

47. One or more cell(s) from a melanocyte culture according to claim 46, which are autologous melanocyte(s).

48. Two or more cells from a melanocyte culture according to claim 46, which are monoploid

49. A medicament comprising one or more cell(s) from a melanocyte culture according to claim 46.

50. A composition comprising one or more cell(s) from a melanocyte culture according to claim 46, for use as a medicament.

51. A pharmaceutical composition for use in an autologous cell implantation comprising one or more cell(s) from a melanocyte culture according to claim 46.

52. A pharmaceutical composition for the treatment of Parkinson's disease in a patient in need thereof comprising one or more cell(s) from a melanocyte culture according to claim 46.

53. A method for screening for substances capable of effecting neuronal cells from a human patient suffering from Parkinson's disease, which method comprises
a) employing a more than 90% pure autologous human melanocyte culture from said patient
b) pre-plating a plate with one or more potentially effective substances,
c) plating one or more cell(s) from said melanocyte culture generated in step a) onto said plate,
d) incubating said melanocytes with said substances during a decided incubation time, and
e) analyzing the plate to identify the substances that display an effect on the plated cell(s).

54. A method according to claim 53, wherein said melanocytes are at least 90% pure, such as at least 91, 92, 93, 94, 95, 96, 97, 98, 99 or 100% pure.

55. A method according to claim 53, wherein said melanocytes are at least 95 to 100% pure.

56. A method according to claim 53, which is executed as a high-throughput screening.

57. A method according to claim 53, wherein the plates in step e) are analysed on a reader such as a fluorescence-reader.

58. A method according to claim 53, wherein the non-viable cell(s) effected by a substance from the plate in step e), are removed, and the viable cells are analysed using a cell counter or flow cytometric analysis.

59. A method according to claim 53, wherein said melanocyte(s) used in said method is/are generated by a method comprising
a) culturing epidermal human cells in a serum-free, pituitary extract free and phorbol ester-free medium in the presence of antibiotics, and
b) subculturing the epidermal cells in the presence of at least 0.75 mM Ca²⁺.

60. A method according to claim 53, further comprising identifying a substance suitable in an individual medical treatment method for a human patient suffering from Parkinson's disease.

61. A substance identified by a method according to claim 53 for treating Parkinson's disease in a human patient.

62. A method for screening for a predisposition for Parkinson's disease in a human patient, comprising testing the sensitivity of one or more cell(s) from an autologous melanocyte culture from said patient for a substance identified by a method according to claim 53.

63. A method for screening for a predisposition for Parkinson's disease in a human patient, comprising testing the sensitivity of one or more cell(s) from an autologous melanocyte culture from a patient predisponary for Parkinson's disease for a test substance, and comparing the sensitivity of said patient's one or more cell(s) from an autologous melanocyte culture, to the sensitivity of one or more cell(s) from an autologous melanocyte culture from a healthy individual.

64. A method according to claim 63, wherein said melanocytes are at least 90% pure, such as at least 90, 91, 92, 93, 94, 95, 96, 97, 98, 99 or 100% pure.

65. A method according to claim 63, wherein said melanocytes are at least 95 to 100% pure.

66. A method according to claim 63, wherein the plates in step e) are analysed on a reader such as a fluorescence-reader.

67. A method according to claim 63, wherein the non-viable cell(s) effected by a substance from the plate in step e), are removed, and the viable cells are analysed using a cell counter or flow cytometric analysis.

68. A method according to claim 63, wherein said melanocyte(s) used in said method is/are generated by a method comprising
a) culturing epidermal human cells in a serum-free, pituitary extract free and phorbol ester-free medium in the presence of antibiotics, and
b) subculturing the epidermal cells in the presence of at least 0.75 mM Ca²⁺.

69. A method for screening for substances capable of effecting neuronal cells from a human patient suffering from Parkinson's disease, which method comprises:
f) employing a more than 90% pure autologous human melanocyte culture from each patient,
g) pre-plating a double set of plates with identical one or more potentially neurotoxic substance(s),
h) plating one or more cell(s) from one of said melanocyte cultures generated in step a) onto each set of plates,
i) incubating said melanocytes with said substances during a decided incubation time, and
j) analysing the plates to identify the effect that the substances have on the plated cell(s), and
k) comparing the sensitivity of the melanocyte cultures from each patient to the substances.

70. A method according to claim 69, wherein said melanocytes are at least 90% pure, such as at least 90, 91, 92, 93, 94, 95, 96, 97, 98, 99 or 100% pure.

71. A method according to claim 69, wherein said melanocytes are at least 95 to 100% pure.

72. A method according to claim 69, wherein the plates in step e) are analysed on a reader such as a fluorescence-reader.

73. A method according to claim 69, wherein the non-viable cell(s) effected by a substance from the plate in step e), are removed, and the viable cells are analysed using a cell counter or flow cytometric analysis.

74. A method according to claim 69, wherein said melanocyte(s) used in said method is/are generated by a method comprising
a) culturing epidermal human cells in a serum-free, pituitary extract free and phorbol ester-free medium in the presence of antibiotics, and
b) subculturing the epidermal cells in the presence of at least 0.75 mM Ca²⁺.
